(19) United States
(12) Patent Application Publication
(10) Pub. No.: US 2025/0318783 A1
(43) Pub. Date: Oct. 16, 2025

(54) WEARABLE DEVICE FOR MEASURING CONTACT PRESSURE AND FIT

(71) Applicant: Oura Health Oy, Oulu (FI)

(72) Inventors: Mika Petteri Kangas, Oulu (FI); Antti Kalevi Lämsä, Oulu (FI)

(21) Appl. No.: 19/095,627

(22) Filed: Mar. 31, 2025

Related U.S. Application Data
(60) Provisional application No. 63/632,434, filed on Apr. 10, 2024.

(51) Int. Cl. A61B 5/00

(52) U.S. Cl. CPC A61B 5/6843; A61B 5/6826; A61B 2562/0247; A61B 2562/12

(57) ABSTRACT

Methods, systems, and devices for manufacturing and operating a wearable device are described. A wearable ring device may include one or more elastically-deformable protrusions extending from an inner curved surface (e.g., inner circumferential surface) of the housing, where the one or more elastically-deformable protrusions are formed via a vacuum molding process (and/or pressure molding process) that is configured to force a moldable material through one or more apertures of the housing. The wearable ring device may further include one or more pressure sensors disposed within the one or more elastically-deformable protrusions, where the one or more pressure sensors are configured to acquire pressure data associated with a deformation of the one or more elastically-deformable protrusions. The pressure data may be associated with a relative pressure or contact between the wearable ring device and a tissue of the user.

### CROSS-REFERENCE

The present application for patent claims the benefit of U.S. Provisional Patent Application No. 63/632,434 by Kangas et al., entitled “WEARABLE DEVICE FOR MEASURING CONTACT PRESSURE AND FIT” and filed Apr. 10, 2024, which is assigned to the assignee hereof and is hereby expressly incorporated by reference herein in its entirety.

### FIELD OF TECHNOLOGY

The following relates to wearable devices and data processing, including a wearable device for measuring contact pressure and fit.

### BACKGROUND

Some wearable devices may be configured to collect data from users associated with temperature, blood pressure, heart rate, and the like. The wearable devices may use one or more sensors to collect the data from the user. In some examples, a pressure exerted by the wearable device against the tissue of the user may affect a quality of the collected data.

### BRIEF DESCRIPTION OF THE DRAWINGS

FIG. 1 illustrates an example of a system that supports a wearable device for measuring contact pressure and fit in accordance with aspects of the present disclosure.

FIG. 2 illustrates an example of a system that supports wearable device for measuring contact pressure and fit in accordance with aspects of the present disclosure.

FIG. 3 shows an example of a wearable device diagram of a wearable device for measuring contact pressure and fit in accordance with aspects of the present disclosure.

FIG. 4 shows a flowchart illustrating methods that support a wearable device for measuring contact pressure and fit in accordance with aspects of the present disclosure.

### DETAILED DESCRIPTION

Wearable devices can be configured to collect physiological data from users to provide users with more information regarding their sleep patterns and overall health. Physiological data collected from wearable devices may include heart rate data, temperature data, respiration rate data, blood oxygen saturation data, and the like. In some cases, the wearable devices may include one or more protrusions that house one or more light emitting diodes (LEDs) or light sensors such as photodetectors (PDs) that may transmit and measure light pulses to collect the physiological data. However, in some cases, light emitted by LEDs of a wearable device may penetrate tissue of the user differently depending on a pressure applied to the measurement area of the tissue. In other words, the penetration depth of the light may be based on the contact pressure between the tissue of the user and the wearable device. Thus, variations in pressure between the wearable device and the tissue (due to tightness of the wearable device, swelling, aging, etc.) may cause variation in the collected physiological data, which may result in less accurate measurements and may reduce user satisfaction. Additionally, if the pressure between the wearable device and the tissue is below a threshold (e.g., if the wearable device is not in contact with the tissue of the user), the wearable device may be unable to collect physiological data from the user.

Some wearable devices may use temperature measurements and/or optical techniques to estimate a level of contact pressure between the user's tissue and the wearable device. For example, optical measurements may be used to detect whether or not the wearable device is against the tissue of the user (e.g., based on an amount of light detectable by sensors of the wearable device). However, such techniques may not provide a pressure measurement that quantifies an amount of pressure exerted by the wearable device against the tissue of the user.

Accordingly, techniques described herein may enable the wearable device to perform a pressure measurement of a pressure exerted by the wearable device against the tissue of the user. In particular, aspects of the present disclosure are directed to using pressure sensors within elastically-deformable protrusions in order to measure a contact pressure (e.g., level of skin contact) between a wearable device and a tissue of a user.

For example, LEDs and PDs of a wearable device may be disposed beneath “domes” (e.g., protrusions) that contact a tissue of the user. In such cases, the dome-shaped protrusions of the wearable device may be formed via a vacuum molding process and/or pressure molding process, where the protrusions are formed via a moldable material (e.g., moldable plastic polymer material) such as acrylic or polymethyl methacrylate (PMMA), polycarbonate (PC), polyethylene terephthalate (PETE or PET), or polyamide (PA)). In some aspects, the protrusions may be hollow (e.g., air-filled, gas-filled) and elastically-deformable, as compared to solid protrusions molded from an epoxy material. In such cases, the wearable device may include one or more pressure sensors disposed within one or more of the protrusions that may measure an air pressure within each of the protrusions (e.g., or within the wearable device as a whole).

For example, a protrusion may deform (e.g., depress inwards towards an inner shell of the wearable device) when a pressure exerted by the wearable device against the tissue of the user increases, which may decrease a volume within the protrusion. Accordingly, an air pressure within the protrusion may increase as the protrusion deforms (e.g., due to the same amount of gas filling a space with a relatively smaller volume than a non-deformed protrusion). Similarly, the air pressure within the protrusion may decrease as the protrusion reforms (e.g., elastically reshapes into a molded dome shape). As such, the wearable device may determine a pressure exerted by the wearable device against the tissue of the user based on the measured air pressure within the deformable protrusion(s).

The pressure data may be used to perform several determinations/functions, including determining a wearing status of the wearable device (e.g., determine whether or not the wearable device is currently being worn), estimating a level of contact between the wearable device and the tissue of the user, determining a relative level of “reliability” or “confidence” associated with a signal or measurement, determining an orientation of the wearable device, adjusting operational parameters and/or an activation status of sensors of the wearable device, adjusting/correcting data acquired by the wearable device, or any combination thereof. For example, the wearable device may therefore determine if a pressure exerted by the wearable device against the tissue of the user is outside of a threshold pressure range for performing physiological measurements. If the pressure is larger than an upper threshold of the threshold pressure range, the wearable device may adjust (e.g., correct) measurements performed by the wearable device to account for relatively low quality of measurements associated with the increased pressure, which may result in relatively higher quality (e.g., accuracy) of collected physiological data. If the pressure is less than a lower threshold of the threshold pressure range, the wearable device may refrain from performing physiological measurements, which may increase a battery life of the wearable device.

Aspects of the disclosure are initially described in the context of systems supporting physiological data collection from users via wearable devices. Aspects of the disclosure are further illustrated by and described with reference to wearable device diagrams, apparatus diagrams, system diagrams, and flowcharts that relate to wearable device for measuring contact pressure and fit.

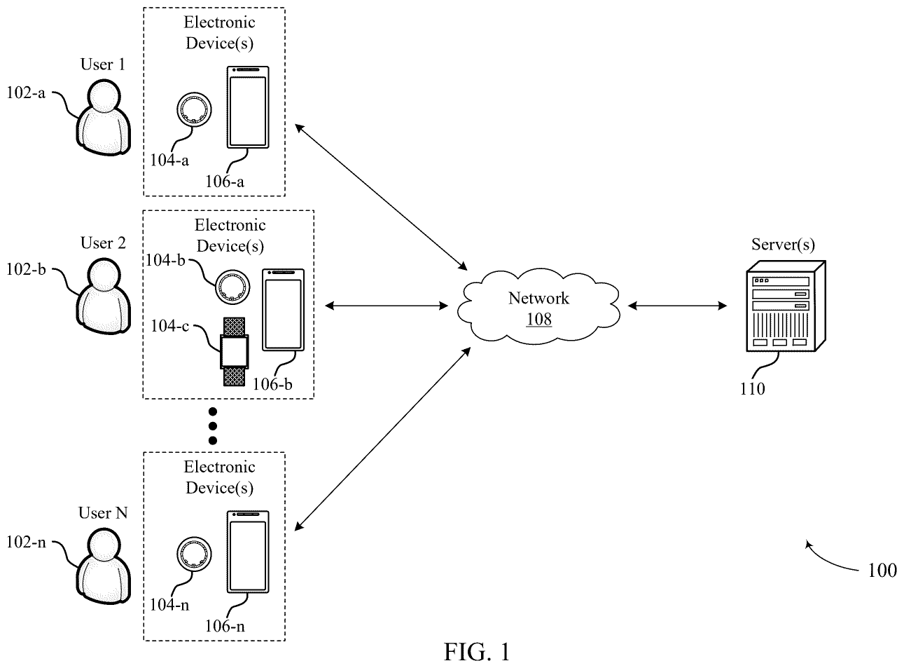

FIG. 1 illustrates an example of a system 100 that supports a wearable device for measuring contact pressure and fit in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled with one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled with the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a wearable device 104 to perform a pressure measurement of a pressure exerted by the wearable device 104 against tissue (e.g., a finger) of a user 102. For example, one or more protrusions (e.g., protrusions housing one or more sensors and/or inductive charging components) of the wearable device 104 may be vacuum formed protrusions (e.g., formed from a moldable plastic polymer material such as PMMA, PC, PET, or PA). The protrusions may therefore be hollow (e.g., air-filled, gas-filled) and elastically-deformable, as compared to solid protrusions molded from an epoxy material. The wearable device 104 may include one or more pressure sensors disposed within one or more of the protrusions that may measure an air pressure within each of the protrusions (e.g., and/or within the wearable device as a whole).

Processors of the wearable device 104 may determine a pressure exerted by the wearable device 104 against the tissue of the user 102 based on the measured air pressure. Using the pressure data, the wearable device 104 may determine whether the wearable device is being worn, estimate a level of contact between the wearable device and the tissue of the user, determine an orientation of the wearable device, adjust operational parameters and/or an activation status of sensors of the wearable device, adjust or correct data acquired by the wearable device, or any combination thereof.

For example, if a pressure exerted by the wearable device 104 against the tissue of the user 102 is outside of a threshold pressure range that is suitable or otherwise usable for performing physiological measurements. If the pressure is larger than an upper threshold of the threshold pressure range, the wearable device 104 may adjust (e.g., correct) measurements performed by the wearable device 104 to account for relatively low quality of measurements associated with the increased pressure, which may result in relatively higher quality (e.g., accuracy) of collected physiological data. If the pressure is less than a lower threshold of the threshold pressure range, the wearable device 104 may refrain from performing physiological measurements, which may increase a battery life of the wearable device 104. For example, low pressure readings may suggest that the wearable device 104 has little or no contact with the tissue of the user, and the wearable device 104 may therefore deactivate sensors of the wearable device 104 to conserve power.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

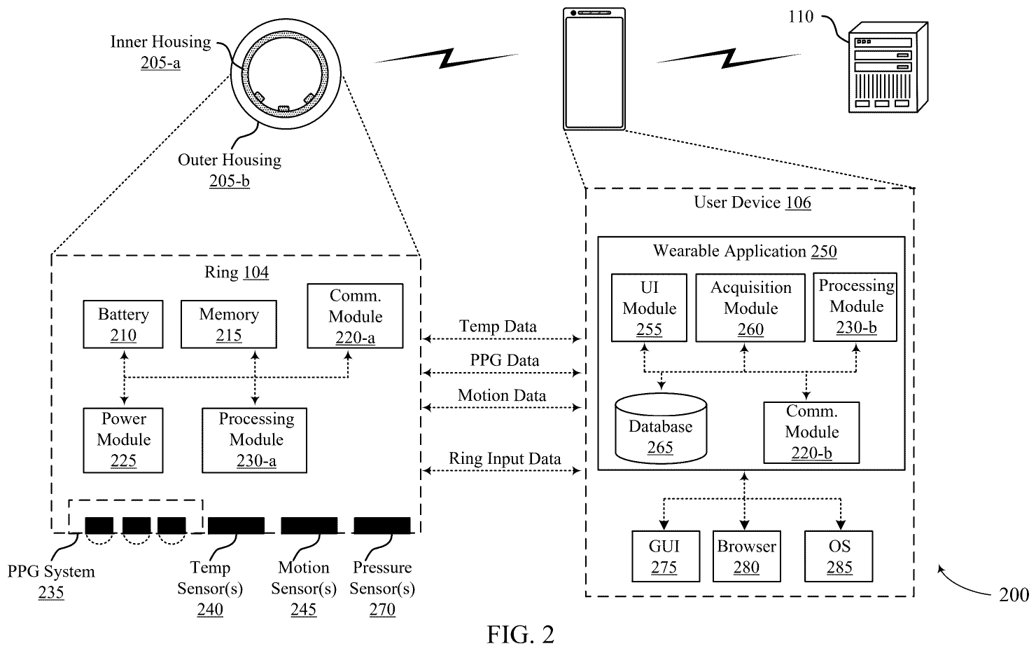

FIG. 2 illustrates an example of a system 200 that supports a wearable device for measuring contact pressure and fit in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for a wearable device 104 to perform a pressure measurement of a pressure exerted by the wearable device 104 against tissue (e.g., a finger) of a user. For example, one or more protrusions (e.g., protrusions housing one or more sensors and/or inductive charging components) of the wearable device 104 may include vacuum formed protrusions (e.g., formed from a moldable plastic polymer material such as PMMA, PC, PET, or PA). The protrusions may therefore be hollow (e.g., air-filled, gas-filled) and elastically-deformable, as compared to solid protrusions molded from an epoxy material. The wearable device 104 may include one or more pressure sensors 270 disposed within the one or more protrusions that may measure an air pressure within each of the protrusions (e.g., or within the wearable device as a whole).

The wearable device 104 may determine a pressure exerted by the wearable device 104 against the tissue of the user based on the measured air pressure. The wearable device may therefore determine if a pressure exerted by the wearable device 104 against the tissue of the user is outside of a threshold pressure range. If the pressure is larger than an upper threshold of the threshold pressure range, the wearable device 104 may adjust (e.g., correct) measurements performed by the wearable device 104 to account for relatively low quality of measurements associated with the increased pressure, which may result in relatively higher quality (e.g., accuracy) of collected physiological data. If the pressure is less than a lower threshold of the threshold pressure range, the wearable device 104 may refrain from performing physiological measurements, which may increase a battery life of the wearable device 104.

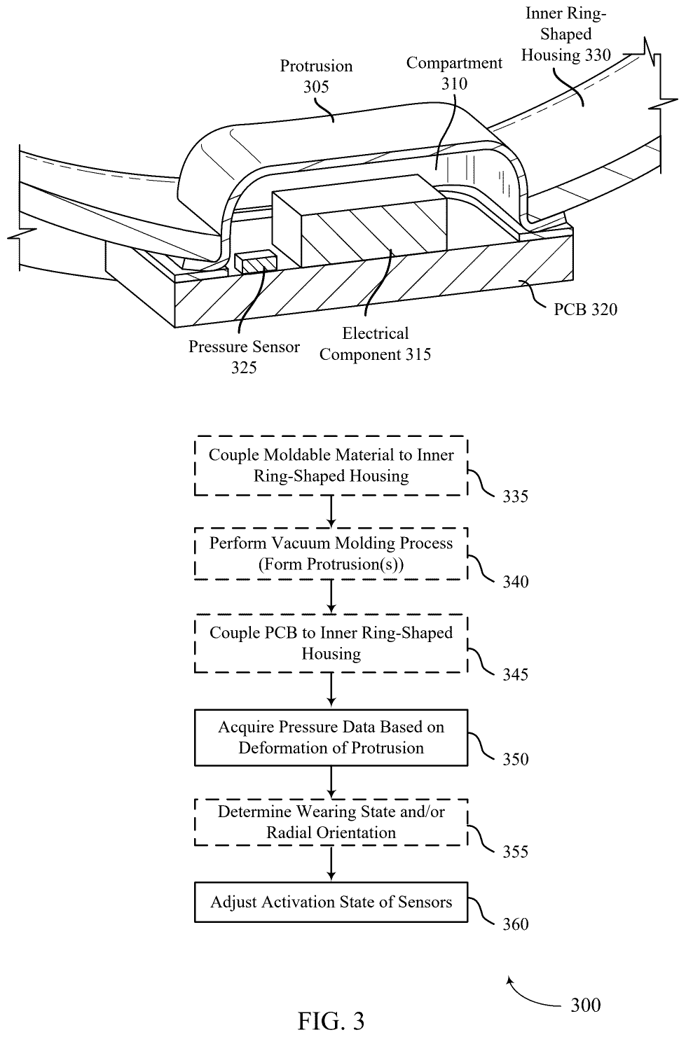

FIG. 3 shows an example of a wearable device diagram 300 that supports a wearable device for measuring contact pressure and fit in accordance with aspects of the present disclosure. The wearable device diagram 300 may implement or may be implemented by one or more aspects of the system 100 and the system 200. For example, the wearable device diagram 300 may illustrate one or more components of (e.g., a cross section of) and operations performed by a wearable ring device, which may be an example of a wearable device 104 as described herein with reference to FIG. 1 and FIG. 2.

In some examples, a wearable device (e.g., a wearable ring device) may include an inner ring-shaped housing 330 with one or more apertures (e.g., apertures radially aligned with one or more electrical components 315 of the wearable device, such as light-emitting components, light-receiving components, or inductive charging components). The inner ring-shaped housing 330 illustrated in FIG. 3 may be an example of the inner housing 205-a illustrated in FIG. 2. The wearable device may include one or more vacuum-formed protrusions 305 (e.g., formed from a moldable plastic polymer material such as PMMA, PC, PET, or PA). The vacuum-formed protrusions 305 may include an optically clear (e.g., transparent or translucent) material or an opaque material. Accordingly, the wearable device may perform one or more physiological measurements (e.g., PPG measurements) and/or charge a rechargeable battery of the wearable device through windows formed by the vacuum-formed protrusions.

In some examples, a manufacturing process for the wearable device may include performing a vacuum molding process (and/or pressure molding process). For example, at step 335, the manufacturing process may include coupling the moldable material with the inner ring-shaped housing 330. For example, the moldable material may be adhered to the inner ring-shaped housing 330 using an adhesive material (e.g., glue).

At 340, the manufacturing process may include performing a vacuum molding process (and/or pressure molding process) to form the protrusions 305. For example, the inner ring-shaped housing 330 and the moldable material may be placed into a mold that will be used to perform the vacuum molding process and/or pressure molding process. A vacuum or other pump may be used to force portions of the moldable material through the one or more apertures of the inner ring-shaped housing 330, thereby forming the one or more protrusions 305 through the apertures. That is, one or more vacuums may apply a vacuum pressure to the moldable material to “suck” the moldable material into the indentations of the mold. For example, the one or more vacuums may cause the moldable material to extrude through the one or more apertures of the inner ring-shaped housing 330. Conversely, one or more pumps may apply a pressure to the moldable material to “push” the moldable material through the apertures of the inner ring-shaped housing 330 into the indentations of the mold.

In some examples, the protrusions 305 may be formed using heat and pressured air (e.g., an air pressure chamber), a heated pressing head (e.g., a heated material that may press the moldable material into the indentations of the mold). In some aspects, the mold may include recesses or other structures that form the respective shape and size of the protrusions 305. As noted previously herein, in some aspects, the protrusions 305 may be elastically deformable such that the protrusions 305 are able to change shape (in response to exerted forces) and return to the previous/original shape without damaging the protrusions 305.

In additional or alternative implementations, the vacuum molding process (and/or pressure molding process) may be performed using a molding plate, where the moldable material is transferred to the inner ring-shaped housing 330 after the molding process. For example, the moldable material may be attached to a molding plate (step 335), where the molding plate may be inserted into a mold to perform the vacuum/pressure molding process (step 340). Following the vacuum/pressure molding process, the moldable material with the formed protrusions 305 may be removed from the mold and from the molding plate, and wrapped around the inner ring-shaped housing 330 such that the protrusions 305 are inserted through the one or more apertures of the inner ring-shaped housing 330. Accordingly, in such cases, the molding plate may include apertures that correspond to the respective apertures of the inner ring-shaped housing 330 such that the protrusions 305 formed through the apertures of the molding plate substantially conform to the apertures of the inner ring-shaped housing 330.

At 345, a PCB 320 may be coupled (e.g., adhered using an adhesive material) to the inner ring-shaped housing 330 (e.g., and the protrusion 305). Accordingly, the manufacturing process may include forming one or more air-filled (e.g., air-tight) compartments 310 between the PCB 320 and the protrusions 305. The compartments 310 may house one or more electrical components 315 (e.g., sensors such as temperature sensors, light-emitting components, and light-receiving components, charging components) usable to acquire physiological measurements from a user. Further, the PCB 320 may include one or more pressure sensors 325 usable to measure a contact pressure between the protrusion 305 (e.g., or one or more other portions of an inner curved and/or circumferential surface of the wearable device) and tissue of the user. An outer ring-shaped housing may be coupled (e.g., adhered using an adhesive material) to the PCB and/or the inner ring-shaped housing 330 to encapsulate the PCB 320 (e.g., and the electrical components 315 and pressure sensors 325) between the inner ring-shaped housing 330, the moldable material, and the outer ring-shaped housing, thereby forming the wearable device.

In some examples, each protrusion 305 of the wearable device may form a separate (e.g., sealed, isolated) compartment 310. For example, the moldable material, the PCB 320, and the adhesive material between the PCB 320 and the inner ring-shaped housing 330 (e.g., or the moldable material) may form an air-tight seal that may prevent air from leaving or entering the compartment 310. Some or all of the compartments 310 of the wearable device may house respective pressure sensors 325. In such examples, a given pressure sensor 325 in a given compartment 310 may measure air pressure within the compartment 310. For example, a first pressure sensor 325 disposed within a first protrusion 305 may measure a first pressure exerted against the tissue of the user in a first radial position along the wearable device 104, and a second pressure sensor 325 disposed within a second protrusion 305 may measure a second pressure exerted against the tissue of the user in a second radial position along the wearable device 104. In some examples, to measure the pressure exerted against the tissue of the user, the pressure sensor may measure a change in air pressure within the compartment 310 as the protrusion 305 deforms. That is, if a relatively larger amount of pressure is exerted against the tissue of the user by the protrusion 305, the protrusion 305 may deform a relatively greater amount into the compartment 310, which may cause the air pressure within the compartment 310 to increase (e.g., as a result of the compartment 310 being air-tight). As such, different protrusions 305/pressure sensors 325 may be configured to estimate the level of contact pressure exerted against the user's tissue at different locations around the wearable device 104, where such information may be used to intelligently control/modify sensors that are used to perform physiological measurements.

Additionally, or alternatively, each protrusion 305 of the wearable device may be coupled with one another such that the protrusions 305 form connected compartments 310. For example, the adhesive material may include one or more channels that may enable air to move between the compartments 310 formed by each protrusion 305. In such examples, one or more pressure sensors 325 (e.g., a pressure sensor 325 in a single compartment 310 of the wearable device, respective pressure sensors 325 in each respective compartment 310 of the wearable device) may measure air pressure within the wearable device as a whole (e.g., a total air pressure within all of the compartments 310 of the wearable device).

At 350, the wearable device may acquire pressure data (e.g., a contact pressure, or lack thereof, between the tissue of the user and the inner curved and/or circumferential surface of the wearable device 104) using the pressure sensors 325. For example, an increase in pressure exerted by the tissue of the user against a respective protrusion 305 (e.g., due to swelling of the appendage of the user) may cause a volume of a respective compartment 310 to decrease (e.g., due to the protrusion 305 elastically deforming), which may increase an air pressure of the compartment 310. Conversely, a decrease in increase in pressure exerted by the tissue of the user against the respective protrusion 305 (e.g., due to removal of the wearable device from the appendage of the user) may cause the volume of a respective compartment 310 to increase, which may decrease an air pressure of the compartment 310. The pressure sensor 325 may accordingly measure a pressure associated with a deformation (of lack thereof) of the protrusion 305. In some examples, if the wearable device includes multiple isolated protrusions 305 with respective pressure sensors 325, the wearable device may acquire first pressure data associated with deformation of a first protrusion 305 and second pressure data associated with deformation of a second protrusion 305.

In some aspects, pressure measurements (e.g., tissue contact pressure measurements) may be performed locally via processors of the wearable device 104. That is, the processors of the wearable device 104 may be configured to acquire the pressure data from the pressure sensors 325 and estimate a contact pressure between the wearable device 104 and the tissue of the user based on the pressure data, and without having to transfer the pressure data to external servers or an external user device 106.

In some examples, at 355, the wearable device may determine a wearing state of the wearable device (e.g., whether or not the wearable device is currently being worn) based on the acquired pressure data. For instance, in cases where the pressure sensors detect an “ambient’ or “baseline” pressure, the wearable device may be configured to determine that there is no deformation of the elastically-deformable protrusions 305, and may therefore determine that the wearable device is not currently being worn by the user. Conversely, in cases where the pressure sensors detect a pressure that is greater than the “ambient’ or “baseline” pressure, the wearable device may be configured to determine that there is some level of deformation of the elastically-deformable protrusions 305, and may therefore determine that the wearable device is currently being worn by the user. In some cases, the wearable device may determine the wearing state (e.g., whether or not the wearable device is currently being worn) based on a combination of data sources, including the pressure data, temperature data (e.g., temperature data acquired via temperature sensors of the wearable device), light-based data (e.g., PPG data acquired via the wearable device), or any combination thereof.

In additional or alternative implementations, the pressure data may be used to determine or estimate a relative level of “reliability” or “confidence” (e.g., reliability metric, confidence value, etc.) associated with received signals and/or measurements performed by the wearable device. The reliability metrics and/or confidence values may be related to (e.g., correlated with) the estimated level of skin contact between the wearable ring device and the tissue of the user (which may also be estimated based on the pressure data). In particular, in cases where the wearable device is able to measure pressure at different protrusions 305, the pressure data collected for the various protrusions may be used to determine the reliability/confidence of signals transmitted and/or received via sensors of each respective protrusion 305. As such, this information may be used to improve channel selection for improved data collection (e.g., select which LEDs and PDs that will be used for data collection).

Additionally, or alternatively, at 355, the wearable device may determine a radial orientation of the wearable device based on the acquired pressure data (and/or other data sources). For example, if a quality of physiological measurements of the user acquired by the wearable device is below a threshold, the wearable device may determine if the lower measurement quality is due to a rotation of the wearable device (e.g., such that the sensors are in a relatively poor position to collect the physiological data, such as over a knuckle of the user) or due to the pressure exerted by the wearable device against the tissue of the user. If the acquired pressure data is outside of a threshold pressure range (e.g., above an upper threshold pressure or below a lower threshold pressure), the wearable device may determine that the lower measurement quality is due to the pressure being outside of the threshold pressure range (e.g., and that the radial orientation of the wearable device is within a threshold of an ideal radial orientation). If the acquired pressure data is inside of the threshold pressure range, the wearable device may determine that the lower measurement quality is due to a rotation of the wearable device into a radial orientation that is a threshold amount outside of the ideal radial orientation.

Additionally, or alternatively, the wearable device may determine the radial orientation of the wearable device 104 based on the first pressure data and the second pressure data. For example, if the first pressure data and the second pressure data indicate that the first protrusion 305 has a greater air pressure than the second protrusion 305, the wearable device may determine that the wearable device has rotated relative to an appendage (e.g., finger) of the user such that a first radial position corresponding to the first protrusion 305 is exerting more pressure against the tissue of the user than a second radial position corresponding to the second protrusion 305.

At 360, the wearable device may perform one or more operations based on the acquired pressure data. For example, the wearable device may adjust operational parameters and/or an activation state of the one or more sensors. In other words, the wearable device may be able to selectively activate/deactivate sensors based on acquired pressure readings, adjust measurement parameters (e.g., applied power, voltages, brightness of LEDs, etc.) used to acquire data, or any combination thereof, based on the acquired pressure data. For instance, if a measured pressure is outside of the threshold pressure range, the wearable device may deactivate one or more light-emitting components and/or light-receiving components, or may determine to adjust (e.g., increase) a brightness of the one or more light-emitting components. In other words, the wearable device 104 may deactivate LEDs/PDs of the wearable device 104 that are located in positions that exhibit relatively poor tissue contact. Similarly, the wearable device 104 may increase a brightness/intensity of LEDs and/or adjust other measurement parameters of sensors (e.g., adjust a current or voltage applied to LEDs) in order to compensate for estimated tissue contact that is outside of some threshold range that is preferred for physiological measurements. If the wearable device determines that the radial orientation of the wearable device is the threshold amount outside of the ideal radial orientation, the wearable device may adjust the activation state of the one or more sensors.

In this regard, by acquiring the pressure data and processing the pressure data locally at the wearable device 104 (e.g., via processors of the wearable device 104), techniques described herein may enable the wearable device 104 to intelligently adjust sensors of the wearable device to improve physiological data collection. For instance, techniques described herein may enable the wearable device 104 to refrain from collecting physiological data when the contact pressure with the user's tissue would result in low quality data (and/or to refrain from “trusting” or using measurements collected when the contact pressure is outside of the threshold pressure range). Additionally, or alternatively, the wearable device may be able to adjust and/or correct acquired physiological data to account for the relative level of contact between the wearable device and the tissue of the user (e.g., increase/decrease a magnitude of measurements, adjust confidence levels of collected data, etc.). As such, techniques described herein may reduce power consumption at the wearable device 104 and improve battery life while simultaneously increasing the overall quality of acquired data. Moreover, techniques described herein may reduce a quantity of physiological data that is collected by the wearable device 104 and/or transmitted to an external device (e.g., user device 106, servers 110), further decreasing the power consumption at the wearable device 104.

Additionally, or alternatively, the wearable device may trigger a graphical user interface (GUI) of a user device paired with the wearable device to display a message to the user. For example, the message may request the user to adjust the radial orientation of the wearable device, or may inform the user that the wearable device is an improper size (e.g., is too large or too small for the appendage of the user).

In some examples, the wearable device 104 may adjust one or more physiological measurements performed by the wearable device 104 based on the acquired pressure data. For example, if the measured pressure is below a lower threshold, the acquired physiological measurements (e.g., blood oxygen saturation (SpO2) measurements) may be different (e.g., relatively less accurate) as compared to physiological measurements performed while the contact pressure is within the threshold pressure range (e.g., due to light from the one or more light-emitting components penetrating the tissue of the user to a different depth). In particular, SpO2 measurements may be sensitive to changing contact pressures. Accordingly, the wearable device 104 may perform one or more calculations to correct the physiological measurements, or may interpret the collected physiological data differently depending on the measured pressure.

Additionally, or alternatively, the wearable device 104 may adjust a configuration of sensors (e.g., an LED configuration) based on the acquired pressure data. For example, the wearable device 104 may select an optical measurement path (e.g., an optical channel between a light-emitting component and a light-receiving component), a measurement duration, an LED pulsing pattern, and the like based on the acquired pressure data. For instance, the wearable device 104 may be configured to reduce a power level of sensors in cases where the contact pressure is within some optimal range for physiological measurements.

In some examples, the wearable device 104 may determine or adjust for an atmospheric pressure based on the acquired pressure data. For example, if an air pressure within one or more compartments 310 increases (e.g., at one or more specified rates of increase or with a regularity that is within a threshold), the wearable device 104 may determine that an atmospheric pressure of the environment surrounding the wearable device 104 is decreasing due to an increase in altitude. Additionally, or alternatively, if the wearable device 104 determines that an atmospheric pressure is changing due to a change in altitude, the wearable device 104 may perform one or more calculations to adjust the acquired pressure data to more accurately represent a contact pressure between the inner curved surface (e.g., inner circumferential surface) of the wearable device 104 and the tissue of the user (e.g., by adjusting for air pressure differences in the compartments 310 due to altitude changes).

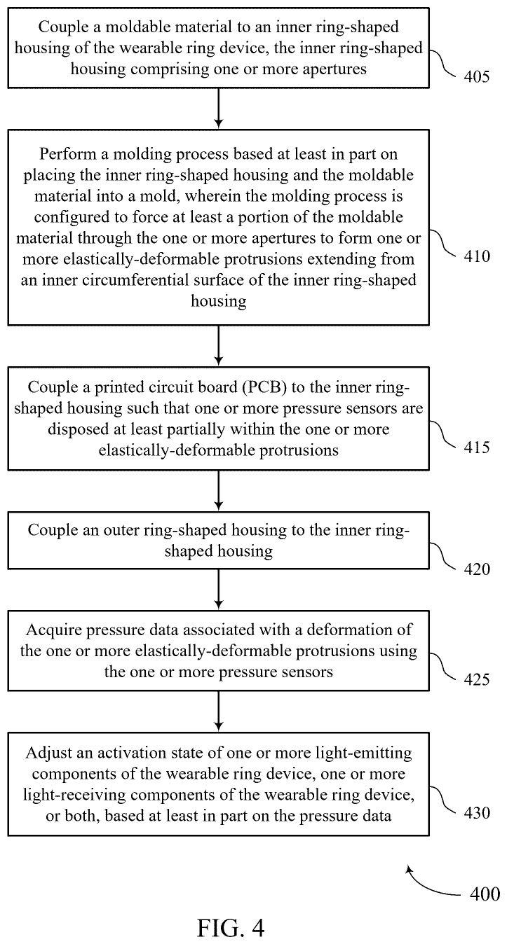

FIG. 4 shows a flowchart illustrating a method 400 that supports a wearable device for measuring contact pressure and fit in accordance with aspects of the present disclosure. The operations of the method 400 may be implemented by a wearable device or its components as described herein. For example, the operations of the method 400 may be performed by a wearable device as described with reference to FIGS. 1 through 3. In some examples, a wearable device may execute a set of instructions to control the functional elements of the wearable device to perform the described functions. Additionally, or alternatively, the wearable device may perform aspects of the described functions using special-purpose hardware.

At 405, the method may include coupling a moldable material to an inner ring-shaped housing of the wearable ring device, the inner ring-shaped housing comprising one or more apertures. The operations of 405 may be performed in accordance with examples as disclosed herein.

At 410, the method may include performing a molding process (e.g., vacuum molding process, pressure molding process) based at least in part on placing the inner ring-shaped housing and the moldable material into a mold, wherein the molding process is configured to force at least a portion of the moldable material through the one or more apertures to form one or more elastically-deformable protrusions extending from an inner curved surface (e.g., inner circumferential surface) of the inner ring-shaped housing. The operations of 410 may be performed in accordance with examples as disclosed herein.

At 415, the method may include coupling a PCB to the inner ring-shaped housing such that one or more pressure sensors are disposed at least partially within the one or more elastically-deformable protrusions. The operations of 415 may be performed in accordance with examples as disclosed herein.

At 420, the method may include coupling an outer ring-shaped housing to the inner ring-shaped housing. The operations of 420 may be performed in accordance with examples as disclosed herein.

At 425, the method may include acquiring pressure data associated with a deformation of the one or more elastically-deformable protrusions using the one or more pressure sensors. The operations of 425 may be performed in accordance with examples as disclosed herein.

At 430, the method may include adjusting an activation state of one or more light-emitting components of the wearable ring device, one or more light-receiving components of the wearable ring device, or both, based at least in part on the pressure data. The operations of 430 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A wearable device is described. The wearable device may include a housing comprising an inner ring-shaped housing and an outer ring-shaped housing, wherein the inner ring-shaped housing comprises a plurality of apertures and defines an inner curved surface (e.g., inner circumferential surface) of the wearable ring device, and wherein the outer ring-shaped housing defines an outer curved surface (e.g., outer circumferential surface) of the wearable ring device, one or more elastically-deformable protrusions disposed within the plurality of apertures and extending from the inner curved/circumferential surface of the inner ring-shaped housing. In some aspects, the one or more elastically-deformable protrusions may be formed via a vacuum molding process that is configured to force a moldable material through one or more apertures of the plurality of apertures. The wearable device may further include a PCB disposed within the housing, a plurality of electrical components disposed on the PCB, the plurality of electrical components comprising, one or more light-emitting components, one or more light-receiving components, or both, disposed on a surface of the PCB and extending through the plurality of apertures, and one or more pressure sensors disposed within the one or more elastically-deformable protrusions, wherein the one or more pressure sensors are configured to acquire pressure data associated with a deformation of the one or more elastically-deformable protrusions.

An apparatus is described. The apparatus may include one or more memories storing processor executable code, and one or more processors coupled with the one or more memories. The apparatus may include a housing comprise an inner ring-shaped housing and an outer ring-shaped housing, wherein the inner ring-shaped housing comprises a plurality of apertures and defines an inner curved surface (e.g., inner circumferential surface) of the wearable ring device, and wherein the outer ring-shaped housing defines an outer curved surface (e.g., outer circumferential surface) of the wearable ring device, one or more elastically-deformable protrusions dispose within the plurality of apertures and extending from the inner curved/circumferential surface of the inner ring-shaped housing, the one or more elastically-deformable protrusions formed via a vacuum molding process that is configured to force a moldable material through one or more apertures of the plurality of apertures, a print circuit board (PCB) disposed within the housing, a plurality of electrical components dispose on the PCB, the plurality of electrical components comprising, one or more light-emit components, one or more light-receiving components, or both, disposed on a surface of the PCB and extending through the plurality of apertures, and one or more pressure sensors dispose within the one or more elastically-deformable protrusions, wherein the one or more pressure sensors are configured to acquire pressure data associated with a deformation of the one or more elastically-deformable protrusions.

In some examples of the method, apparatus, and non-transitory computer-readable medium described herein, the one or more processors may be configured to and adjust an activation state of the one or more light-emitting components, the one or more light-receiving components, or both, based at least in part on the pressure data.

In some examples of the method, apparatus, and non-transitory computer-readable medium described herein, the one or more elastically-deformable protrusions comprise a first elastically-deformable protrusion and a second elastically-deformable protrusion that may be isolated from the first elastically-deformable protrusion and the one or more pressure sensors comprise a first pressure sensor disposed within the first elastically-deformable protrusion and a second pressure sensor disposed within the second elastically-deformable protrusion.

In some examples of the method, apparatus, and non-transitory computer-readable medium described herein, the first elastically-deformable protrusion may be disposed at a first radial position along the inner curved/circumferential surface of the wearable ring device and the second elastically-deformable protrusion may be disposed at a second radial position along the inner curved/circumferential surface of the wearable ring device that may be different from the first radial position.

In some examples of the method, apparatus, and non-transitory computer-readable medium described herein, one or more processors communicatively coupled with the one or more light-emitting components, the one or more light-receiving components, the first pressure sensor, and the second pressure sensor, wherein the one or more processors may be configured to receive first pressure data from the first pressure sensor disposed at the first radial position, and second pressure data from the second pressure sensor disposed at the second radial position, and determine a radial orientation of the wearable ring device relative to an appendage of a user.

Some examples of the method, apparatus, and non-transitory computer-readable medium described herein may further include operations, features, means, or instructions for adjusting an activation state of the one or more light-emitting components, the one or more light-receiving components, or both, based at least in part on the radial orientation of the wearable ring device.

In some examples of the method, apparatus, and non-transitory computer-readable medium described herein, the one or more elastically-deformable protrusions comprise a first elastically-deformable protrusion and a second elastically-deformable protrusion that may be coupled with one another and the one or more pressure sensors may be configured to acquire the pressure data associated with the deformation of the first elastically-deformable protrusion, the second elastically-deformable protrusion, or both.

In some examples of the method, apparatus, and non-transitory computer-readable medium described herein, the one or more light-emitting components, the one or more light-receiving components, or both, may be disposed at least partially within the one or more elastically-deformable protrusions.

In some examples of the method, apparatus, and non-transitory computer-readable medium described herein, the pressure data may be associated with a contact pressure between a tissue of a user and the inner curved/circumferential surface of the inner ring-shaped housing.

In some examples of the method, apparatus, and non-transitory computer-readable medium described herein, the one or more elastically-deformable protrusions form at least a portion of a boundary of one or more air-tight compartments and the one or more pressure sensors may be disposed at least partially within the one or more air-tight compartments.

In some examples of the method, apparatus, and non-transitory computer-readable medium described herein, the PCB forms at least a second portion of the boundary of the one or more air-tight compartments.

A method for manufacturing and using a wearable ring device by an apparatus is described. The method may include coupling a moldable material to an inner ring-shaped housing of the wearable ring device, the inner ring-shaped housing comprising one or more apertures, performing a vacuum molding process based at least in part on placing the inner ring-shaped housing and the moldable material into a mold, wherein the vacuum molding process is configured to force at least a portion of the moldable material through the one or more apertures to form one or more elastically-deformable protrusions extending from an inner curved surface (e.g., inner circumferential surface) of the inner ring-shaped housing, coupling a PCB to the inner ring-shaped housing such that one or more pressure sensors are disposed at least partially within the one or more elastically-deformable protrusions, coupling an outer ring-shaped housing to the inner ring-shaped housing, acquiring pressure data associated with a deformation of the one or more elastically-deformable protrusions using the one or more pressure sensors, and adjusting an activation state of one or more light-emitting components of the wearable ring device, one or more light-receiving components of the wearable ring device, or both, based at least in part on the pressure data.

An apparatus for manufacturing and using a wearable ring device is described. The apparatus may include one or more memories storing processor executable code, and one or more processors coupled with the one or more memories. The one or more processors may individually or collectively be operable to execute the code to cause the apparatus to couple a moldable material to an inner ring-shaped housing of the wearable ring device, the inner ring-shaped housing comprising one or more apertures, perform a vacuum molding process based at least in part on placing the inner ring-shaped housing and the moldable material into a mold, wherein the vacuum molding process is configured to force at least a portion of the moldable material through the one or more apertures to form one or more elastically-deformable protrusions extending from an inner curved surface (e.g., inner circumferential surface) of the inner ring-shaped housing, couple a PCB to the inner ring-shaped housing such that one or more pressure sensors are disposed at least partially within the one or more elastically-deformable protrusions, couple an outer ring-shaped housing to the inner ring-shaped housing, acquire pressure data associated with a deformation of the one or more elastically-deformable protrusions using the one or more pressure sensors, and adjust an activation state of one or more light-emitting components of the wearable ring device, one or more light-receiving components of the wearable ring device, or both, based at least in part on the pressure data.

Another apparatus for manufacturing and using a wearable ring device is described. The apparatus may include means for coupling a moldable material to an inner ring-shaped housing of the wearable ring device, the inner ring-shaped housing comprising one or more apertures, means for performing a vacuum molding process based at least in part on placing the inner ring-shaped housing and the moldable material into a mold, wherein the vacuum molding process is configured to force at least a portion of the moldable material through the one or more apertures to form one or more elastically-deformable protrusions extending from an inner curved surface (e.g., inner circumferential surface) of the inner ring-shaped housing, means for coupling a PCB to the inner ring-shaped housing such that one or more pressure sensors are disposed at least partially within the one or more elastically-deformable protrusions, means for coupling an outer ring-shaped housing to the inner ring-shaped housing, means for acquiring pressure data associated with a deformation of the one or more elastically-deformable protrusions using the one or more pressure sensors, and means for adjusting an activation state of one or more light-emitting components of the wearable ring device, one or more light-receiving components of the wearable ring device, or both, based at least in part on the pressure data.

A non-transitory computer-readable medium storing code for manufacturing and using a wearable ring device is described. The code may include instructions executable by one or more processors to couple a moldable material to an inner ring-shaped housing of the wearable ring device, the inner ring-shaped housing comprising one or more apertures, perform a vacuum molding process based at least in part on placing the inner ring-shaped housing and the moldable material into a mold, wherein the vacuum molding process is configured to force at least a portion of the moldable material through the one or more apertures to form one or more elastically-deformable protrusions extending from an inner curved surface (e.g., inner circumferential surface) of the inner ring-shaped housing, couple a PCB to the inner ring-shaped housing such that one or more pressure sensors are disposed at least partially within the one or more elastically-deformable protrusions, couple an outer ring-shaped housing to the inner ring-shaped housing, acquire pressure data associated with a deformation of the one or more elastically-deformable protrusions using the one or more pressure sensors, and adjust an activation state of one or more light-emitting components of the wearable ring device, one or more light-receiving components of the wearable ring device, or both, based at least in part on the pressure data.

In some examples of the method, apparatus, and non-transitory computer-readable medium described herein, the one or more elastically-deformable protrusions comprise a first elastically-deformable protrusion and a second elastically-deformable protrusion that may be isolated from the first elastically-deformable protrusion and the one or more pressure sensors comprise a first pressure sensor disposed within the first elastically-deformable protrusion and a second pressure sensor disposed within the second elastically-deformable protrusion.

In some examples of the method, apparatus, and non-transitory computer-readable medium described herein, the first elastically-deformable protrusion may be disposed at a first radial position along the inner curved/circumferential surface of the wearable ring device and the second elastically-deformable protrusion may be disposed at a second radial position along the inner curved/circumferential surface of the wearable ring device that may be different from the first radial position.

Some examples of the method, apparatus, and non-transitory computer-readable medium described herein may further include operations, features, means, or instructions for receiving first pressure data from the first pressure sensor disposed at the first radial position, and second pressure data from the second pressure sensor disposed at the second radial position and determining a radial orientation of the wearable ring device relative to an appendage of a user.

In some examples of the method, apparatus, and non-transitory computer-readable medium described herein, the activation state of the one or more light-emitting components, the one or more light-receiving components, or both, may be adjusted based at least in part on the radial orientation of the wearable ring device.

In some examples of the method, apparatus, and non-transitory computer-readable medium described herein, the one or more elastically-deformable protrusions comprise a first elastically-deformable protrusion and a second elastically-deformable protrusion that may be coupled with one another and the one or more pressure sensors may be configured to acquire the pressure data associated with the deformation of the first elastically-deformable protrusion, the second elastically-deformable protrusion, or both.

In some examples of the method, apparatus, and non-transitory computer-readable medium described herein, the one or more light-emitting components, the one or more light-receiving components, or both, may be disposed at least partially within the one or more elastically-deformable protrusions.

In some examples of the method, apparatus, and non-transitory computer-readable medium described herein, the pressure data may be associated with a contact pressure between a tissue of a user and the inner curved/circumferential surface of the inner ring-shaped housing.

In some examples of the method, apparatus, and non-transitory computer-readable medium described herein, the one or more elastically-deformable protrusions form at least a portion of a boundary of one or more air-tight compartments and the one or more pressure sensors may be disposed at least partially within the one or more air-tight compartments.

In some examples of the method, apparatus, and non-transitory computer-readable medium described herein, the PCB forms at least a second portion of the boundary of the one or more air-tight compartments.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a housing comprising an inner ring-shaped housing and an outer ring-shaped housing, wherein the inner ring-shaped housing comprises one or more apertures and defines at least a portion of an inner curved surface of the wearable ring device, and wherein the outer ring-shaped housing defines at least a portion of an outer curved surface of the wearable ring device;
one or more elastically-deformable protrusions disposed within the one or more apertures and extending from the inner curved surface of the inner ring-shaped housing;
a printed circuit board (PCB) disposed within the housing; and
a plurality of electrical components disposed on the PCB, the plurality of electrical components comprising:
one or more light-emitting components, one or more light-receiving components, or both, disposed on a surface of the PCB and configured to transmit or receive light through the one or more apertures; and
one or more pressure sensors disposed within the one or more elastically-deformable protrusions, wherein the one or more pressure sensors are configured to acquire pressure data associated with a deformation of the one or more elastically-deformable protrusions.

2. The wearable ring device of claim 1, further comprising:
one or more processors communicatively coupled with the one or more light-emitting components, the one or more light-receiving components, and the one or more pressure sensors, wherein the one or more processors are configured to:
adjust an activation state of the one or more light-emitting components, the one or more light-receiving components, or both, based at least in part on the pressure data.

3. The wearable ring device of claim 1, wherein the one or more elastically-deformable protrusions comprise a first elastically-deformable protrusion and a second elastically-deformable protrusion that is isolated from the first elastically-deformable protrusion, and wherein the one or more pressure sensors comprise a first pressure sensor disposed within the first elastically-deformable protrusion and a second pressure sensor disposed within the second elastically-deformable protrusion.

4. The wearable ring device of claim 3, wherein the first elastically-deformable protrusion is disposed at a first radial position along the inner curved surface of the wearable ring device, and wherein the second elastically-deformable protrusion is disposed at a second radial position along the inner curved surface of the wearable ring device that is different from the first radial position.

5. The wearable ring device of claim 4, further comprising:
one or more processors communicatively coupled with the one or more light-emitting components, the one or more light-receiving components, the first pressure sensor, and the second pressure sensor, wherein the one or more processors are configured to:
receive first pressure data from the first pressure sensor disposed at the first radial position, and second pressure data from the second pressure sensor disposed at the second radial position; and
determine a radial orientation of the wearable ring device relative to an appendage of a user.

6. The wearable ring device of claim 5, wherein the one or more processors are further configured to:
adjust an activation state of the one or more light-emitting components, the one or more light-receiving components, or both, based at least in part on the radial orientation of the wearable ring device.

7. The wearable ring device of claim 1, wherein the one or more elastically-deformable protrusions comprise a first elastically-deformable protrusion and a second elastically-deformable protrusion that are coupled with one another, wherein the one or more pressure sensors are configured to acquire the pressure data associated with the deformation of the first elastically-deformable protrusion, the second elastically-deformable protrusion, or both.

8. The wearable ring device of claim 1, wherein the one or more light-emitting components, the one or more light-receiving components, or both, are disposed at least partially within the one or more elastically-deformable protrusions.

9. The wearable ring device of claim 1, wherein the pressure data is associated with a contact pressure between a tissue of a user and the inner curved surface of the inner ring-shaped housing.

10. The wearable ring device of claim 1, wherein the one or more elastically-deformable protrusions form at least a portion of a boundary of one or more air-tight compartments, wherein the one or more pressure sensors are disposed at least partially within the one or more air-tight compartments.

11. The wearable ring device of claim 10, wherein the PCB forms at least a second portion of the boundary of the one or more air-tight compartments.

12. A method for manufacturing and using a wearable ring device, comprising:
coupling a moldable material to an inner ring-shaped housing of the wearable ring device, the inner ring-shaped housing comprising one or more apertures;
performing a molding process based at least in part on placing the inner ring-shaped housing and the moldable material into a mold, wherein the molding process is configured to force at least a portion of the moldable material through the one or more apertures to form one or more elastically-deformable protrusions extending from an inner curved surface of the inner ring-shaped housing;
coupling a printed circuit board (PCB) to the inner ring-shaped housing such that one or more pressure sensors are disposed at least partially within the one or more elastically-deformable protrusions;
coupling an outer ring-shaped housing to the inner ring-shaped housing;
acquiring pressure data associated with a deformation of the one or more elastically-deformable protrusions using the one or more pressure sensors; and
adjusting an activation state of one or more light-emitting components of the wearable ring device, one or more light-receiving components of the wearable ring device, or both, based at least in part on the pressure data.

13. The method of claim 12, wherein the one or more elastically-deformable protrusions comprise a first elastically-deformable protrusion and a second elastically-deformable protrusion that is isolated from the first elastically-deformable protrusion, and wherein the one or more pressure sensors comprise a first pressure sensor disposed within the first elastically-deformable protrusion and a second pressure sensor disposed within the second elastically-deformable protrusion.

14. The method of claim 13, wherein the first elastically-deformable protrusion is disposed at a first radial position along the inner curved surface of the wearable ring device, and wherein the second elastically-deformable protrusion is disposed at a second radial position along the inner curved surface of the wearable ring device that is different from the first radial position.

15. The method of claim 14, further comprising:
receiving first pressure data from the first pressure sensor disposed at the first radial position, and second pressure data from the second pressure sensor disposed at the second radial position; and
determining a radial orientation of the wearable ring device relative to an appendage of a user, wherein the activation state of the one or more light-emitting components, the one or more light-receiving components, or both, is adjusted based at least in part on the radial orientation of the wearable ring device.

16. The method of claim 12, wherein the one or more elastically-deformable protrusions comprise a first elastically-deformable protrusion and a second elastically-deformable protrusion that are coupled with one another, wherein the one or more pressure sensors are configured to acquire the pressure data associated with the deformation of the first elastically-deformable protrusion, the second elastically-deformable protrusion, or both.

17. The method of claim 12, wherein the one or more light-emitting components, the one or more light-receiving components, or both, are disposed at least partially within the one or more elastically-deformable protrusions.

18. The method of claim 12, wherein the pressure data is associated with a contact pressure between a tissue of a user and the inner curved surface of the inner ring-shaped housing.

19. The method of claim 12, wherein the one or more elastically-deformable protrusions form at least a portion of a boundary of one or more air-tight compartments, wherein the one or more pressure sensors are disposed at least partially within the one or more air-tight compartments.

20. A method, comprising:
acquiring, via one or more processors of a wearable device, pressure data collected using one or more pressure sensors of the wearable device, the one or more pressure sensors disposed at least partially within one or more elastically-deformable protrusions extending from a surface of the wearable device, the pressure data associated with a deformation of the one or more elastically-deformable protrusions;
determining, using the one or more processors of the wearable device, whether the wearable device is being worn by a user; and
adjusting, using the one or more processors of the wearable device, an activation state of one or more electrical components of the wearable device based at least in part on determining whether the wearable device is being worn by the user.
